(19) United States
(12) Patent Application Publication
(10) Pub. No.: US 2026/0232304 A1
(43) Pub. Date: Aug. 13, 2026

(54) OCCLUDER LOCKING MECHANISMS

(71) Applicant: St. Jude Medical, Cardiology Division, Inc., St. Paul, MN (US)

(72) Inventors: Brian Perszyk, Shoreview, MN (US); Alex Bloomquist, Mound, MN (US); Andrea Osberghaus, New Brighton, MN (US); Tracee Eidenschink, Wayzata, MN (US); Erika Beek, Bloomington, MN (US); Philip Osterbauer, Wyoming, MN (US)

(73) Assignee: St. Jude Medical, Cardiology Division, Inc., St. Paul, MN (US)

(21) Appl. No.: 19/631,429

(22) Filed: Mar. 27, 2026

Related U.S. Application Data
(62) Division of application No. 17/165,738, filed on Feb. 2, 2021.
(60) Provisional application No. 62/969,557, filed on Feb. 3, 2020.

(51) Int. Cl. A61B 17/00

(52) U.S. Cl. CPC A61B 17/0057; A61B 2017/00579; A61B 2017/00592; A61B 2017/00619; A61B 2017/00623; A61B 2017/00632

(57) ABSTRACT

A medical device including a locking mechanism and a method including activating the locking mechanism are described herein. The medical device includes distal and proximal disc portions and a locking mechanism. The locking mechanism is configured to pull and maintain the distal and proximal disc portions toward each other when the medical device is deployed in an expanded configuration.

### CROSS-REFERENCE TO RELATED APPLICATIONS

The present application is a divisional of U.S. Patent Application No. 17/165,738, filed Feb. 2, 2021, which claims the benefit of priority to U.S. Provisional Patent Application No. 62/969,557, filed Feb. 3, 2020, the entire contents of both of which are hereby incorporated by reference in their entirety.

### BACKGROUND OF THE DISCLOSURE

### Field of Disclosure

The present disclosure relates generally to medical devices used in the human body, such as those that occlude undesired blood flow. In particular, the present disclosure is directed to locking mechanisms incorporated into medical devices delivered to a target site within the human body. More specifically, the present disclosure is directed to active and passive locking mechanisms that may reduce damage to cardiac tissue.

### Background

A wide variety of medical devices are used to treat any target site, such as an abnormality, a vessel, an organ, an opening, a chamber, a channel, a hole, a cavity, or the like, located anywhere in the body. Many known devices, including medical devices having at least one disc (e.g., devices having one disc and one lobe or devices having two discs) and configured to clamp in place upon deployment at the target site, are made of Nitinol material. In order to provide sufficient clamping and radial force to overcome forces from the anatomy, the designs with Nitinol can become radially stiff to achieve the desired clamping forces and meet other criteria such as the shape memory properties and delivery needs. For example, most devices that occlude undesired blow flow include one disc and a waist section, or two discs in a disc–waist–disc configuration. Devices having at least one disc, such as those configured to occlude left atrial appendage (LAA), atrial septal defect (ASD), and patent foramen ovale (PFO), may benefit from radially softer devices and/or improved clamping force.

Thus, a relatively softer device with minimal radial disc force and maximum disc deformation/conformability (e.g., especially around the superior aspect of the atrium near the aortic root) would serve to increase device compliance on the tissue and thereby minimize the risk of tissue erosion. However, when a softer frame/braid material is used, the anatomy has a greater effect on the device shape. For example, with a softer device it may be necessary to oversize the device in order to get sufficient clamping force when anchoring the device, and consequently at least one disc of the device may bulge due to increased compression. The bulging effect of the Nitinol frame may occur especially with thicker septa and increased oversizing of the device relative to the space being occluded. When bulging is minimized, a softer frame conforms to the anatomy better, which may improve occlusion effectiveness.

One way to combat the bulging is to hold the center of the disks together after deployment via a locking mechanism. In the rare case of embolization, the device may have to be snared and recaptured. When the device is snared, the locking mechanism either has to be reversible, or weak enough that pulling the device into a catheter will release/uncouple the mechanism.

Accordingly, it would be desirable to provide locking mechanisms on medical devices that minimize bulging of the medical device when deployed, thereby minimizing radial disc forces, maximizing disc deformation and conformability, and ultimately improving occlusive effectiveness while reducing damage to cardiac tissue. The locking mechanisms may be active or passive, and reversible or non-reversible, depending on the treatment needs of the medical device at the target site.

### SUMMARY OF THE DISCLOSURE

In one embodiment, the present disclosure is directed to a medical device for treating a target site. The medical device comprises a tubular member and a locking mechanism. The tubular member comprises a proximal disc portion at a proximal end, a distal disc portion at a distal end, and a waist member extending between the proximal disc portion and the distal disc portion. The tubular member has an expanded configuration when deployed at the target site and a reduced configuration for delivery to the target site. The locking mechanism comprises a distal locking portion attached to the distal disc portion and a proximal locking portion attached to the proximal disc portion. The distal locking portion and the proximal locking portion are configured to be coupled together when the medical device is in the expanded configuration.

In another embodiment, the present disclosure is directed to a medical device for treating a target site. The medical device comprises a tubular member and a locking mechanism. The tubular member comprises a proximal disc portion at a proximal end, a distal disc portion at a distal end, and a waist member extending between the proximal disc portion and the distal disc portion. The tubular member has an expanded configuration when deployed at the target site and a reduced configuration for delivery to the target site. The locking mechanism comprises at least one coupling element attached to both the distal disc portion and the proximal disc portion. The at least one coupling element is a spring that internally extends from the distal disc portion to the proximal disc portion in a criss-cross pattern such that the distal disc portion and the proximal disc portion are configured to pull toward each other when the medical device is in the expanded configuration.

In yet another embodiment, the present disclosure is directed to a method of eliminating or reducing erosion of cardiac tissue. The method comprises providing a medical device comprising a tubular member and a locking mechanism. The tubular member comprises a proximal disc portion at a proximal end, a distal disc portion at a distal end, and a waist member extending between the proximal disc portion and the distal disc portion. The tubular member has an expanded configuration when deployed at the target site and a reduced configuration for delivery to the target site. The locking mechanism comprises a distal locking portion attached to the distal disc portion and a proximal locking portion attached to the proximal disc portion. The distal locking portion and the proximal locking portion are configured to be coupled together when the medical device is in the expanded configuration. The method further comprises constraining the medical device in the reduced configuration, delivering the medical device, and deploying the medical device such that the tubular member transitions from the reduced configuration to the expanded configuration. The method also comprises activating the locking mechanism by coupling together the distal locking portion and the proximal locking portion, and increasing the medical device compliance on cardiac tissue.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is an exemplary embodiment of a known medical device in accordance with the present disclosure.

FIG. 2A is an exemplary embodiment of a locking mechanism system with internally threaded end screws and a friction element in accordance with the present disclosure. FIG. 2B is another exemplary embodiment of a locking mechanism system with internally threaded end screws and a friction element in accordance with the present disclosure. FIG. 2C is an exemplary embodiment of a locking mechanism system with internally threaded end screws and a catch element in accordance with the present disclosure. FIG. 2D is an exemplary embodiment of a locking mechanism system with internally threaded end screws and a textured element in accordance with the present disclosure. FIG. 2E is another exemplary embodiment of a locking mechanism system with internally threaded end screws and a textured element in accordance with the present disclosure. FIG. 2F is yet another exemplary embodiment of a locking mechanism system with internally threaded end screws and a textured element in accordance with the present disclosure.

FIG. 3A is an exemplary embodiment of a locking mechanism system in accordance with the present disclosure. FIG. 3B is an exemplary embodiment of the locking mechanism system shown in FIG. 3A with external barbs and threading in accordance with the present disclosure.

FIG. 4A is an exemplary embodiment of a locking mechanism system with a wire loop and latch in a reduced configuration prior to deployment in accordance with the present disclosure. FIG. 4B is an exemplary embodiment of a locking mechanism system with a wire loop and latch in an expanded configuration during deployment in accordance with the present disclosure. FIG. 4C is an exemplary embodiment of a locking mechanism system with a wire loop and latch after deployment in accordance with the present disclosure.

FIG. 5A is an exemplary embodiment of a locking mechanism system with end caps and a suture loop in accordance with the present disclosure. FIG. 5B is another exemplary embodiment of a locking mechanism system with end caps and a suture loop in accordance with the present disclosure.

FIG. 6A is an exemplary embodiment of a locking mechanism system with an engagement rod and receptacle in accordance with the present disclosure. FIG. 6B is an exemplary embodiment of the engagement rod shown in FIG. 6A in accordance with the present disclosure. FIG. 6C is an exemplary embodiment of the locking mechanism system with an engagement rod and receptacle shown in FIG. 6A upon deployment in accordance with the present disclosure.

FIG. 7A is an exemplary embodiment of a locking mechanism system with formed loops and an end screw prior to deployment in accordance with the present disclosure. FIG. 7B is an exemplary embodiment of the locking mechanism system shown in FIG. 7A upon deployment in accordance with the present disclosure. FIG. 7C is an exemplary embodiment of the locking mechanism system shown in FIG. 7A after deployment in accordance with the present disclosure.

FIG. 8A is an exemplary embodiment of a locking mechanism system with an end screw and a coil in accordance with the present disclosure. FIG. 8B is an exemplary embodiment of the locking mechanism system with an end screw and a coil shown in FIG. 8A in a reduced configuration in accordance with the present disclosure. FIG. 8C is an exemplary embodiment of a flat clover shaped spring locking mechanism in accordance with the present disclosure. FIG. 8D is an exemplary embodiment of a spring side profile view before heat setting in accordance with the present disclosure. FIG. 8E is an exemplary embodiment of a spring top view before heat setting in accordance with the present disclosure. FIG. 8F is an exemplary embodiment a spring side view after heat setting and flattening in accordance with the present disclosure. FIG. 8G is an exemplary embodiment of a deployed occluder device without a locking mechanism in accordance with the present disclosure. FIG. 8H is an exemplary embodiment of a deployed occluder device with a spring coupling element as a locking mechanism in accordance with the present disclosure. FIG. 8I is another exemplary embodiment of a deployed occluder device with a spring coupling element as a locking mechanism in accordance with the present disclosure.

FIG. 9A is an exemplary embodiment of a locking mechanism system with an internal elastomer in accordance with the present disclosure. FIG. 9B is an exemplary embodiment of a locking mechanism system with an internal elastomer in a reduced configuration in accordance with the present disclosure. FIG. 9C is an exemplary embodiment of an internal view of an elastomer spring coupling element when loaded into an occluder device in an un-deployed, semi-expanded configuration in accordance with the present disclosure. FIG. 9D is an exemplary embodiment of a top view of an elastomer spring coupling element in an occluder device having no disc portion coverings and in a flattened configuration in accordance with the present disclosure. FIG. 9E is an exemplary embodiment of the occluder device of FIG. 9D with PET blood blocking discs inserted into distal and proximal disc portions in accordance with the present disclosure.

Corresponding reference characters indicate corresponding parts throughout the several views of the drawings. It is understood that the Figures are not necessarily to scale.

### DETAILED DESCRIPTION OF THE DISCLOSURE

The present disclosure generally relates to center locking mechanisms incorporated into medical devices for treating a target site. The present disclosure discloses medical devices having locking mechanisms configured to pull a distal disc portion and a proximal disc portion towards each other to minimize bulging of the disc portions. Accordingly, the medical devices of the present disclosure enable minimized bulging of the medical device when deployed, thereby also minimizing radial disc forces, maximizing disc deformation and conformability, and ultimately improving occlusive effectiveness while reducing damage to cardiac tissue. The locking mechanisms may be active or passive, and reversible or non-reversible, depending on the treatment needs of the medical device at the target site.

The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

It is understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an LAA, an atrial septal defect (ASD), a lesion, a vessel dissection, or a tumor. Embodiments of the medical device may be useful, for example, for occluding an LAA, ASD, ventricular septal defect (VSD), or patent ductus arteriosus (PDA), as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator, and the term “distal” refers to a part of the medical device or the delivery device that is farther from the operator at any given time as the medical device is being delivered through the delivery device.

The medical device may include one or more layers of occlusive material, wherein each layer may be comprised of any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial time period. In exemplary embodiments of the medical device described herein, the occlusive material (not shown) is attached to a frame of the occlusive device to close or restrict access (e.g., of bodily fluids such as blood) through a passageway (or access passage) of the occlusive medical device. In this way, the occlusive material ensures the medical device performs its occlusive function, as described above herein. Each layer of material is formed from an occlusive, yet penetrable material, such that access through the passageway of the occlusive medical device by other medical devices is not restricted. In the exemplary embodiment, a “penetrable” material is more easily punctured, separated, slit, pierced, or otherwise penetrated than the material that forms the frame.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

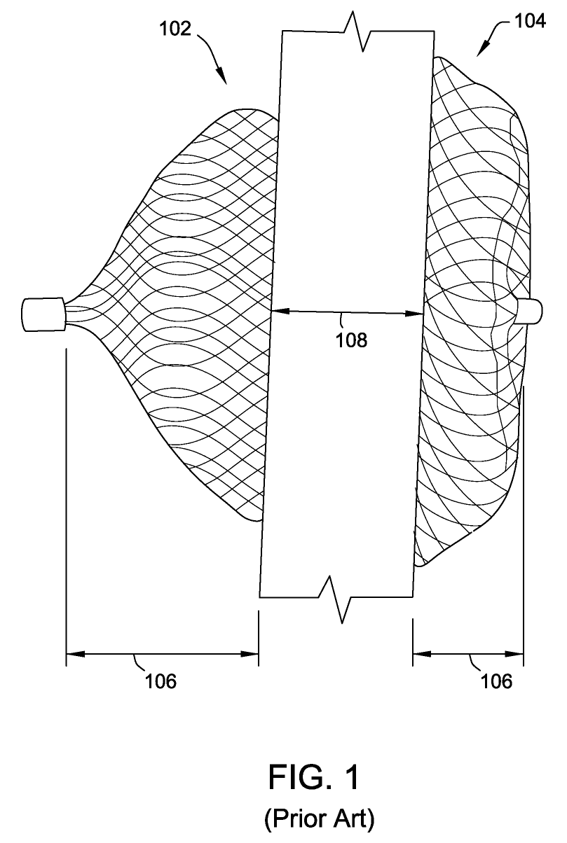

In at least some conventional or known medical devices, such as a medical device having a distal disc portion 102 and a proximal disc portion 104 shown in FIG. 1, significant bulging 106 of both disc portions occurs when the medical device is deployed through a septum of thickness 108. Bulging 106 may occur with medical devices that are relatively softer due to the anatomy having a greater effect on a shape of the device than would occur with medical devices that are relatively stiffer. While a relatively stiffer device (e.g., such as a device made with a relatively stiff Nitinol braid) provides sufficient clamping and radial forces to overcome forces from the anatomy and also returns to its formed shape upon deployment/implant, the stiffer device may not adequately conform to the anatomy (particularly e.g., with larger thicknesses 108 at the septum) such that the risk of tissue erosion increases. As described above, when softer medical devices are deployed, bulging 106 may occur and may compromise the occlusion effectiveness of the medical device.

The medical devices of the present disclosure, which include both active and passive locking mechanisms to pull together and further to maintain a pulled-together configuration of the distal and proximal disc portions, avoid at least these disadvantages of known medical devices.

### Active Locking Mechanisms

In an exemplary embodiment, the locking mechanism is an active locking mechanism. An active locking mechanism requires manual coupling of a distal locking portion (attached to a distal disc portion) to a proximal locking portion (attached to a proximal disc portion) when in the expanded configuration. Prior to deployment of the medical device, the distal disc portion and proximal disc portion are not initially coupled. During deployment, an additional step of activating the locking mechanism must be executed in order to couple the distal and proximal disc portions to each other such that the distal and proximal disc portions pull toward each other when in the expanded configuration and such that the distal and proximal disc portions maintain their pulled-together configuration after deployment is complete. In an exemplary embodiment, the distal locking portion and the proximal locking portion are secured together by the manual coupling required by the active locking mechanism.

In an exemplary embodiment, the distal locking portion is located at a center of the distal disc portion and likewise the proximal locking portion is located at a center of the proximal disc portion. Alternatively, the distal locking portion may be located anywhere on the distal disc portion and the proximal locking portion may be located anywhere on the proximal disc portion. In an exemplary embodiment, the distal and proximal locking portions are attached to their respective disc portions such that they are enabled to be coupled together to minimize bulging of the device.

Active locking mechanisms may be reversible or non-reversible. In a reversible embodiment, the locking mechanism is reversible such that when the distal locking portion and the proximal locking portion are uncoupled from each other, the distal locking portion remains attached to the distal disc portion and the proximal locking portion remains attached to the proximal disc portion. In some embodiments, the locking mechanism may be considered reversible if, after being retrieved from the target site within the body, the device can immediately be re-deployed (to a same or new target site within the body). Alternatively, the locking mechanism may be non-reversible such that when the distal locking portion and the proximal locking portion are uncoupled from each other, at least one of the distal locking portion and the proximal locking portion detaches from its respective disc portion. In some embodiments, the locking mechanism may be considered non-reversible if the device cannot be immediately re-deployed within the body after being retrieved from the target site.

### a. Internally threaded end screws with friction element

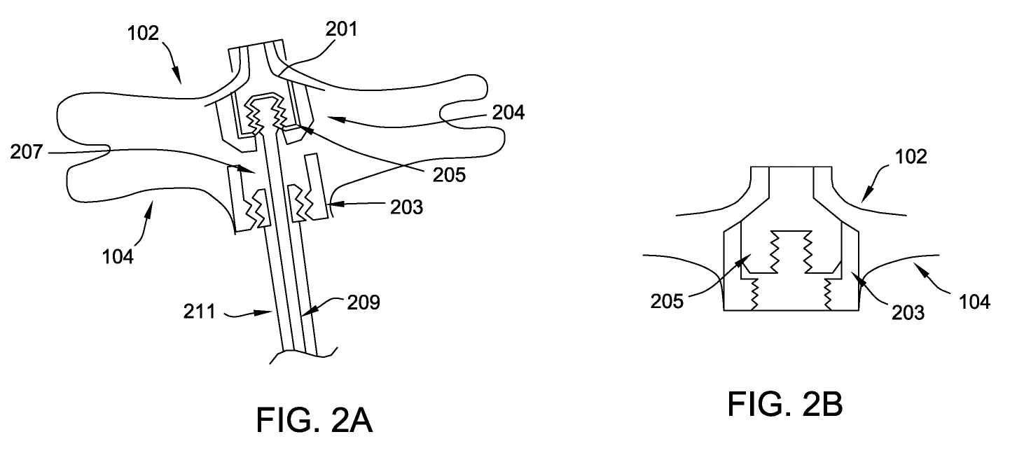

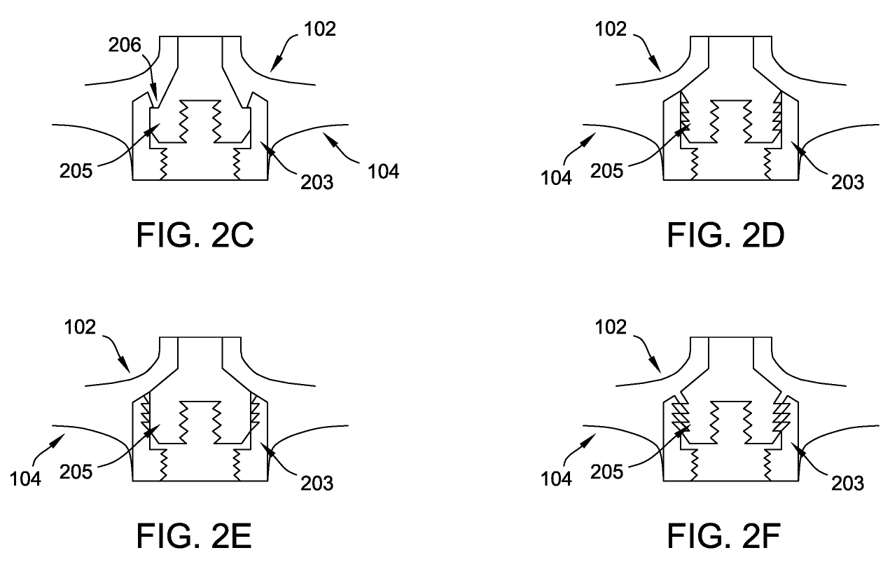

Turning now to FIGS. 2A and 2B, embodiments are shown in which a locking mechanism system includes a distal locking portion 201, a proximal locking portion 203, and a coupling element 205. As shown in the illustrated embodiment, distal locking portion 201 and proximal locking portion 203 are both internally threaded end screws. Known devices, such as Abbott’s Amplatzer^{TM} devices, have used proximal screw mechanisms for release of the device from a delivery cable to complete deployment. In this embodiment, the locking mechanism to secure the center of the disc portions together utilizes a smaller end screw 201 (e.g., 0000-160 or smaller) for the distal disc portion 102 to pull it into proximal locking portion 203 (e.g., a larger proximal end screw) of the proximal disc portion (not shown) and fixing the two together via coupling element 205.

Proximal locking portion 203 includes a pocket-type receptacle 207 for receiving coupling element 205. Depending on the embodiment, coupling element 205 may be a friction element, a catch element, and/or a textured element. FIG. 2A shows coupling element 205 as a friction element further comprising compressible sleeve (e.g., an adhered or over-molded polymer compressible sleeve) 204 which will conform to fit into pocket-type receptacle 207 during deployment and remain locked after deployment owing to frictional forces between compressible sleeve 204 and proximal locking portion 203. FIG. 2A further shows a distal cable 209 having an externally threaded distal end that screws into the internally threaded area of distal locking portion 201, and a proximal delivery cable 211 having an externally threaded distal end that screws into the internally threaded area of proximal locking portion 203. Proximal delivery cable 211 also includes a through lumen for distal cable 209. During deployment, distal cable 209 is used to pull distal locking portion 201 (with coupling element 205 attached) toward proximal locking portion 203 until coupling element 205 is secured within pocket-type receptacle 207, thus activating the locking mechanism. In some embodiments, during deployment coupling element 205 and pocket-type receptacle 207 of proximal locking portion 203 may be visible by imaging in order to show they are engaged prior to release of delivery cables.

FIG. 2B is another embodiment showing coupling element 205 as a friction element. In this embodiment, coupling element 205 itself is made of a softer material than proximal locking portion 203, such that coupling element 205 enables a friction fit within pocket-type receptacle 207 and forms the locking mechanism for the device. In some embodiments, coupling element 205 may be a friction element such as a metallic split compression ring or a spring loop.

The embodiment of FIG. 2C shows a locking mechanism between coupling element 205 and proximal locking portion 203 enabled by a catch element 206. In some embodiments, catch element 206 may be formed by a flexible proximal locking portion 203 such that pocket-type receptacle 207 expands to accept coupling element 205, and subsequently distal edge of flexible proximal locking portion 203 may be slightly taller than coupling element 205, such that flexible proximal locking portion 203 shrinks back down around coupling element 205 and slipping is minimized or eliminated. In other embodiments, catch element 206 may be established by a formed lip on a distal inner edge of proximal locking portion 203 that is configured to catch or grasp over a radially protruding edge of coupling element 205.

Turning now to FIGS. 2D, 2E, and 2F, a locking mechanism includes a textured element for at least a portion of the engagement surfaces of proximal locking portion 203, coupling element 205, or both. A textured element may include a surface comprising teeth, barbs, fingers, and combinations thereof, as well as other suitable textured surfaces that would enable proximal locking portion 203 and coupling element 205 to become and remain engaged. FIG. 2D shows coupling element 205 only having a textured outer surface to engage pocket-type receptacle 207 of proximal locking portion 203. FIG. 2E shows proximal locking portion 203 only having a textured inner surface that forms an outer edge of pocket-type receptacle 207. FIG. 2F shows both coupling element 205 and proximal locking portion 207 as having textured surfaces configured to engage with one another to form a locking mechanism for the device.

After securing the two disc portions according to any of the locking mechanisms described herein above, the distal locking portion 201 (shown in FIG. 2A) on the distal disc portion 102 is released from distal cable 209, and distal cable 209 is pulled through a lumen of the proximal delivery cable 211. Then proximal delivery cable 211 is released from proximal locking portion 203, thus releasing it from the device. In the case of embolization, coupling element 205 would uncouple from the proximal disc portion 104 (not shown) during recapture, and if it was desirable, the device could be reused by reattaching the end screw locking portions to their respective disc portions.

### b. External barbs and threading

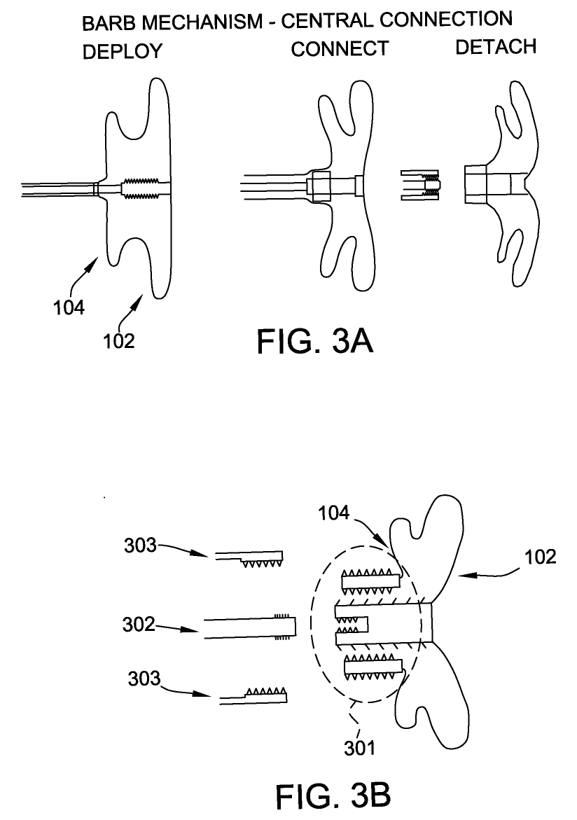

FIGS. 3A and 3B show an exemplary embodiment of a locking mechanism system. FIG. 3A shows a barb mechanism central connection locking mechanism when ready to deploy (left), during deployment when barbed locking mechanism connects and locks distal disc portion 102 and proximal disc portion 104 together (center), and after detachment of delivery cables (right). FIG. 3B depicts distal locking portion 301 including a plurality of barbs, and proximal delivery cable 303 as well as distal delivery cable 302 each including a plurality of threaded members configured to engage the plurality of barbs of distal locking portion 301. In some embodiments, distal locking portion 301 may additionally include internal threading for delivery cable attachment. In some embodiments, distal locking portion 301 may include external threading configured to engage with a plurality of barbs included on proximal delivery cable 303. A combination of barbs and threading enable adjustment of the locking mechanism to optimize coupling of the distal and proximal disc portions and compliance of the device to the tissue (e.g., based on anatomy). In an exemplary embodiment, the locking mechanism allows at least one of a loosened configuration and a tightened configuration between the distal locking portion and the proximal and distal delivery cables (303, 302) when coupled.

### c. Wire loop and latch/fastener

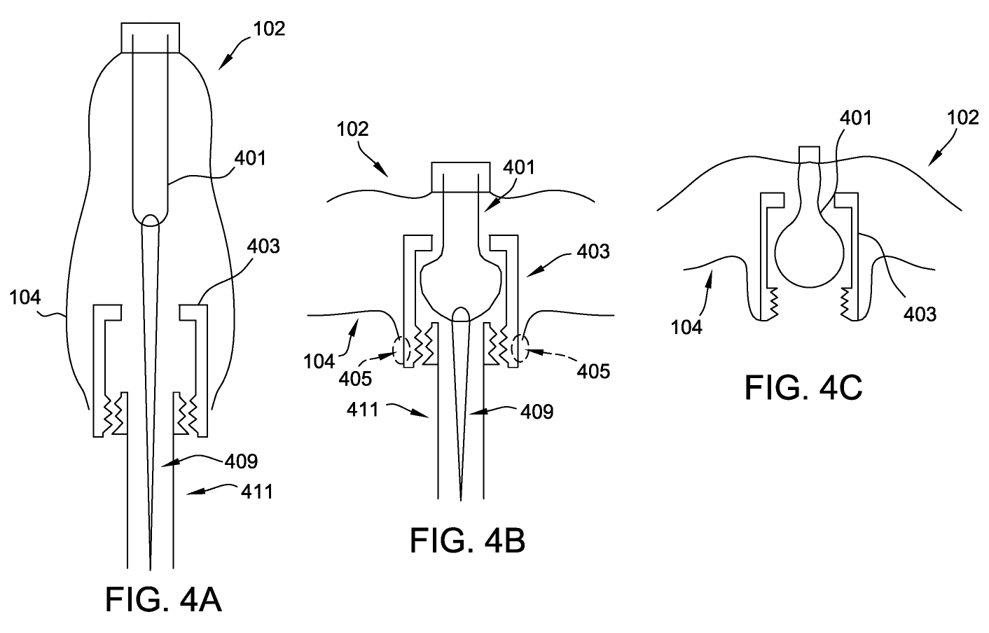

FIGS. 4A, 4B, and 4C show embodiments of a locking mechanism system prior to deployment, during deployment, and after deployment, respectively. Distal locking portion 401 comprises a wire loop and proximal locking portion 403 comprises an internally threaded end screw with a distally-extended latch-type fastener configured to engage an expanded configuration of the wire loop of distal locking portion 401. Proximal locking portion 403 may be attached to proximal disc 104 at attachment point 405 (shown within dashed circle), e.g., by welding or bonding. The delivery system can be attached to the loop of distal locking portion 401 with a removable tether 409 (or a clasping bioptome-like mechanism). Prior to deployment (FIG. 4A) the device is in a reduced configuration for loading into delivery system (not shown) and wire loop of distal locking portion 401 may have a smaller/narrower profile within the device. As shown in FIG. 4A, wire loop of distal locking portion 401 is elongated and under tension such that the attached removable tether 409 extends distally through proximal locking portion 403. During deployment (FIG. 4B), the device is foreshortened into an expanded configuration such that the elongated wire loop of distal locking portion 401 is positioned within the latch-type fastener of proximal locking portion 403. Because the tension on distal locking portion 401 is decreased when the device is transitioned from the reduced configuration to the expanded configuration, wire loop of distal locking portion 401 may begin to expand within the latch-type fastener of proximal locking portion 403 in order to engage proximal locking portion 403 as the locking mechanism for the device. Removable tether 409 may be used to pull distal disc portion 102 toward both proximal disc portion 104 and proximal locking portion 403 until wire loop of distal locking portion 401 has more fully expanded, thus enabling distal locking portion 401 to be securely engaged by the latch-type fastener of proximal locking portion 403. That is, the foreshortened and expanded wire loop of distal locking portion 401 is securely coupled within proximal locking portion 403. Once the device is deployed (FIG. 4C), tension is released from distal locking portion 401, allowing the wire loop of distal locking portion 401 to fully expand with proximal locking portion 403. This fully expanded wire loop engages the locking mechanism and prevents the two discs from bulging in the deployed configuration. In some embodiments, tether 409 may be removed, followed by removal of proximal delivery cable 411 to release the device (FIG. 4C). In some embodiments, distal locking portion 401 and proximal locking portion 403 are able to fully couple/lock together securely only once removable tether 409 is released/removed. In some embodiments, the connection between removable tether 409 and distal locking portion 401 is maintained after deployment (though tension has been released) to enable retrieval or repositioning of the device.

### d. Suture loop and end caps

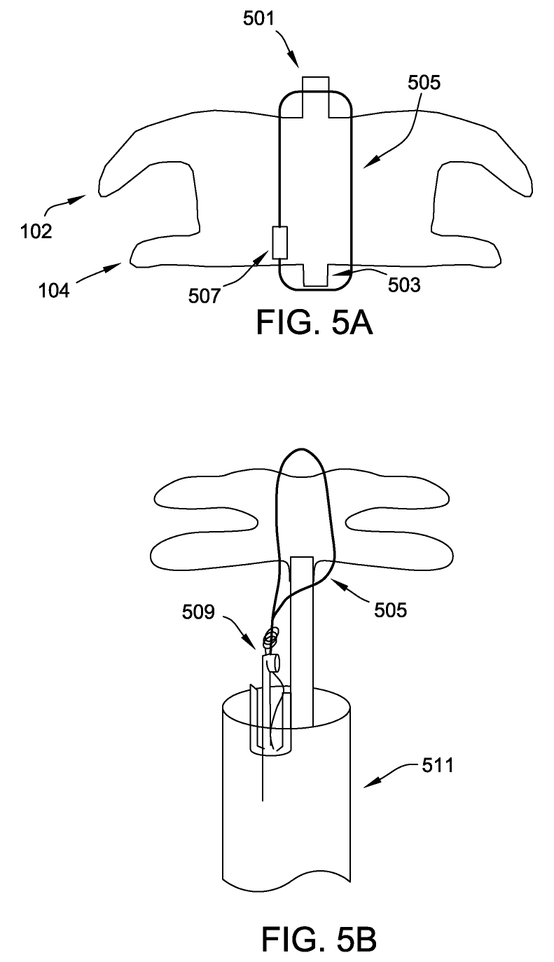

FIGS. 5A and 5B show exemplary embodiments of a suture loop coupling element 505 and distal and proximal locking portion end caps, 501 and 503. In an exemplary embodiment, a noose-type loop coupling element 505 (e.g., a slip-knotted suture) is introduced via a distal cable/tether 509 through a proximal delivery cable 511 (e.g., a ‘tube inside a tube’ type delivery system) and is looped around a distal locking portion 501 endcap as well as a proximal locking portion 503 endcap. In some embodiments, noose-type loop coupling element 505 is a large single loop that extends through both distal disc portion 102 and proximal disc portion 104 and is connected with a slip knot. The single loop is both large enough and loose enough for the device to be longitudinally pulled into the delivery system. As the device is delivered/deployed, noose-type loop coupling element 505 will foreshorten and the loop will remain long until tension is pulled on the slip knot, upon which the loop will shorten and lock both discs together. In some embodiments, tether 509 may be used when loading and recapturing the device, and an additional mechanism 507 (such as a bioptome-type mechanism) may be needed to push the knot, cinch the loop adequately on the device, and cut off any excess suture prior to releasing the device. In some embodiments, the slip-knotted suture is the coupling element 505 and is pre-assembled on the device and is loose, so as not to interfere with the Nitinol shape formation, and when proximal and distal disc portions (104, 102) are formed the knot in the delivery system is pushed down and cinched. Then the tube in tube that is over the suture can be twisted or pushed to cut the suture leaving the knot with device. In an exemplary embodiment, shown in FIG. 5A and 5B, a long suture is used to extend out of the body to be released/cut to remove the long tether, which is shown in FIG. 5B. For example, a push rod is used to slide the knot down and cinch the loop tight. FIG. 5B shows a depiction of how to cut or release the long suture loop. Depending on treatment requirements of the device, use of a suture or suture-type material may be less desirable (than a wire loop as shown in FIG. 4, for example) because these locking mechanism embodiments are non-reversible since the suture must be cut/severed upon recapture.

### e. Engagement rod and receptacle

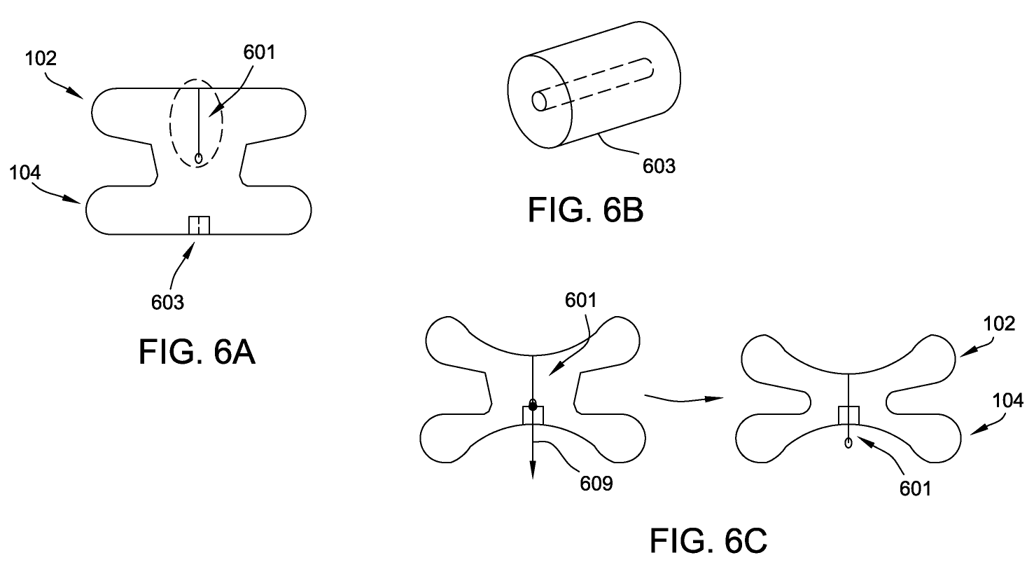

Turning now to FIGS. 6A, 6B, and 6C, an embodiment is shown of a device locking mechanism that includes an engagement rod distal locking portion 601 (shown within the dashed oval) and a proximal locking portion 603 receptacle (FIG. 6A). FIG. 6B is an enlarged view of proximal locking portion 603 having a small central channel configured for receiving engagement rod distal locking portion 601 in order to engage the locking mechanism for the device. During deployment (FIG. 6C), removable tether 609 is used to pull distal disc portion 102 in the direction of the arrow toward proximal disc portion 104 via the attachment to engagement rod distal locking portion 601. The disc portions are coupled once engagement rod distal locking portion 601 is securely positioned within receptacle of proximal locking portion 603. Either one or both of distal locking portion 601 and proximal locking portion 603 may be formed using materials that are sticky, stretchable, textured (e.g., flocked), and/or have a high coefficient of friction (e.g., 10-50 Shore A silicone) to enable adequate coupling. Friction due to the smaller size/diameter of the receptacle relative to the larger size/diameter of the engagement rod may additionally or alternatively affect success coupling/locking of distal disc portion 102 to proximal disc portion 104. In some embodiments, distal locking portion 601 is an engagement rod that has an eyelet or a slightly enlarged proximal end to allow tether attachment and to improve secure attachment once engaged with proximal locking portion 603.

In some embodiments, the locking mechanism comprises a plurality of locking mechanisms. The plurality of locking mechanisms comprises a plurality of distal locking portions evenly distributed over the distal disc portion and a plurality of proximal locking portions evenly distributed over the proximal disc portion such that each of the plurality of distal locking portions is configured to be coupled to a respective one of the plurality of proximal locking portions. Alternatively, the plurality of distal and proximal locking portions may be unequally distributed over their respective disc portion; however each of the plurality of distal locking portions should be configured to be coupled to a respective one of the plurality of proximal locking portions.

### f. Methods of using the device

In accordance with the present disclosure, the medical devices disclosed herein are directed toward methods of eliminating or reducing erosion of cardiac tissue. The methods comprise providing a medical device comprising a tubular member comprising a proximal disc portion at a proximal end and a distal disc portion at a distal end and a waist member extending between the proximal disc portion and the distal disc portion; wherein the tubular member has an expanded configuration when deployed at the target site and a reduced configuration for delivery to the target site; and, at least one locking mechanism; constraining the medical device from a preset expanded configuration to a reduced configuration; delivering the medical device; deploying the medical device such that the tubular member returns to the preset expanded configuration; activating the locking mechanism by coupling together the distal locking portion and the proximal locking portion; and, increasing the medical device compliance on cardiac tissue.

### Passive Locking Mechanisms

In an exemplary embodiment, the locking mechanism is a passive locking mechanism. A passive locking mechanism automatically couples the distal locking portion and the proximal locking portion when in the expanded configuration. Prior to deployment of the medical device, the distal disc portion and proximal disc portion are coupled and remain coupled in both reduced and expanded configurations of the device. For example, the locking mechanism (or a portion of the locking mechanism) may be in a stretched and/or elongated state to accommodate the reduced configuration, which then tightens and/or shortens when the device is transitioned to the expanded configuration upon deployment. The tightened and/or shortened state of the locking mechanism serves to pull together the distal and proximal disc portions and further maintains the pulled together configuration of the disc portions in the expanded configuration of the device.

In an exemplary embodiment, the distal locking portion is located at a center of the distal disc portion and likewise the proximal locking portion is located at a center of the proximal disc portion. Alternatively, the distal locking portion may be located anywhere on the distal disc portion and the proximal locking portion may be located anywhere on the proximal disc portion. In an exemplary embodiment, the distal and proximal locking portions are attached to their respective disc portions such that they are enabled to be coupled together to minimize bulging of the device.

Passive locking mechanisms may be reversible or non-reversible. In a reversible embodiment, the locking mechanism is reversible such that when the distal locking portion and the proximal locking portion are uncoupled from each other, the distal locking portion remains attached to the distal disc portion and the proximal locking portion remains attached to the proximal disc portion. In some embodiments, the locking mechanism may be considered reversible if, after being retrieved from the target site within the body, the device can immediately be re-deployed (to a same or new target site within the body). For example, when a locking mechanism (or a portion of the locking mechanism) stretches and/or elongates to accommodate a reduced configuration of the device, and returns to a tightened and/or shortened state to pull the distal and proximal disc portions together (and keep them pulled together) in the expanded state of the device, then the locking mechanism is reversible since the device is immediately re-deployable. Alternatively, the locking mechanism may be non-reversible such that when the distal disc portion and the proximal disc portion are uncoupled from each other, at least one of the distal locking portion and the proximal locking portion detaches from its respective disc portion. In some embodiments, the locking mechanism may be considered non-reversible if the device cannot be immediately re-deployed within the body after being retrieved from the target site. For example, when a locking mechanism (or a portion of a locking mechanism) must be severed in order to uncouple the distal and proximal disc portions from each other, the locking mechanism may be considered non-reversible since the device is no longer immediately re-deployable.

### a. Formed loops and end screw

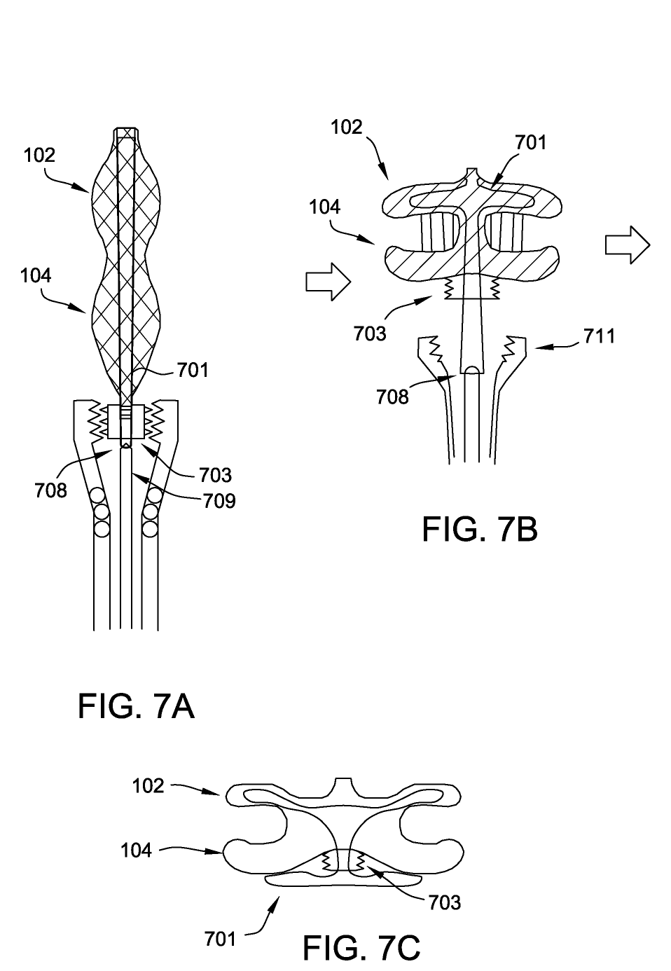

FIGS. 7A, 7B, and 7C show exemplary embodiments of a locking mechanism system prior to deployment, upon deployment, and after deployment, respectively. The locking mechanism system includes distal locking portion 701 comprising at least one formed loop and proximal locking portion 703 comprising at least one formed loop and an externally threaded end screw. Prior to deployment, the at least one formed loop of distal locking portion 701 is attached at tether point 708 to distal cable/removeable tether 709 (FIG. 7A). During deployment when transitioning from the reduced configuration to the expanded configuration and when delivery system’s proximal delivery cable 711 is released, a first loop (of distal locking portion 701) starts to form, as shown in FIG. 7B. Depending on the embodiment, proximal delivery cable may or may not need to flare radially outward in order to reach a desired diameter for engaging internal threads of delivery cable 711 with external threads of proximal disc portion 104. During deployment, distal locking portion 701 is still attached to distal cable/removeable tether 709 at tether point 708, such that formed loop of distal locking portion 701 is still long enough to enable elongated (reduced configuration) delivery of the device. Once formed loop of distal locking portion 701 is foreshortened, it takes on a shape within distal disc portion 102 that pulls both discs together by forming a second loop on the exterior of proximal disc portion 104. The second loop (of distal locking portion 701) forms upon foreshortening of the first loop (which is not released from distal disc portion 102) and removal of distal cable/removable tether 709 and the disc portions are pulled together, as shown in FIG. 7C. In some embodiments, at least one of the formed loops (or locking loops) is both internal and external to the device.

In an exemplary embodiment, the distal disc portion 102 and first loop deploy together and seat against the left atrium, the proximal disc portion 104 seats against the right atrium with the second loop still in the delivery cable 711 (FIG. 7B). Cable 711 detaches from proximal disc portion 104 and finally the second loop is unsheathed from delivery cable 711 to seat against the device (FIG. 7C).

In some embodiments, there is only one formed loop (or locking wire, e.g., a Nitinol wire) pulled by a tether/floss, and it will form a “U” shape allowing the original discs to deploy. If there is bulging, the release of the floss of the U-shaped wire will allow the locking loop to form a distal loop internal to the device and a proximal loop external to the device. These loops are needed to allow for the stretch elongation of the braided implant during delivery. A loop in the locking mechanism is needed because the ratio of length in delivery to final compressed length could be as much as 10:1. The proximal loop external to the device will act like a push rod external to the proximal disc closing the gap.

### b. Spring and end screw

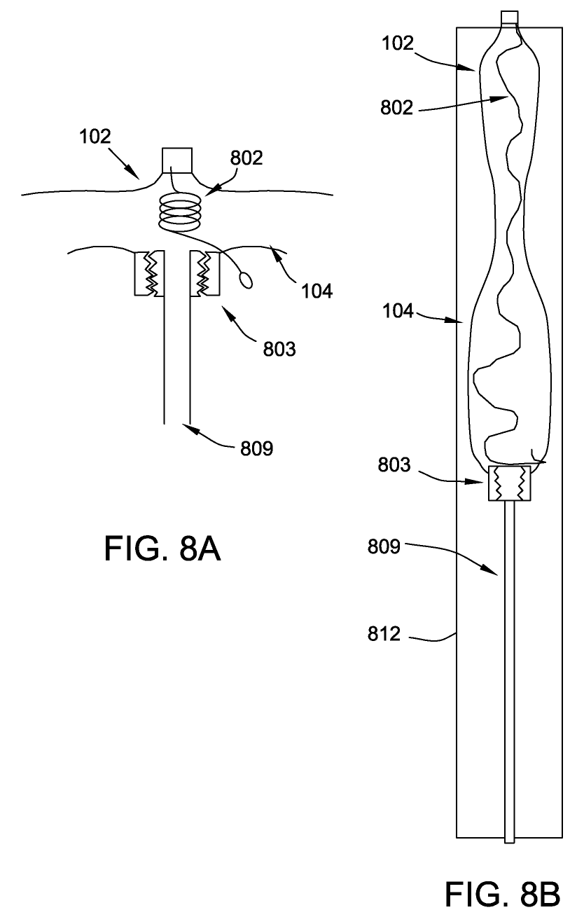

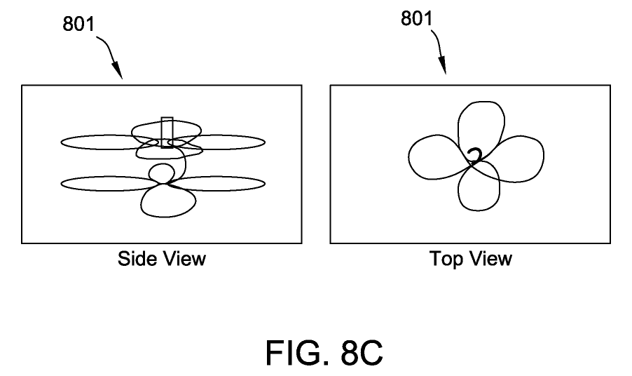

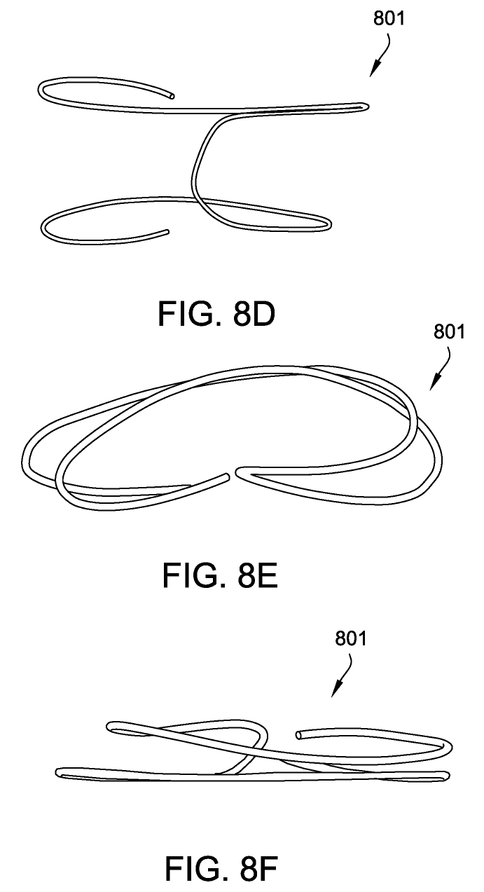

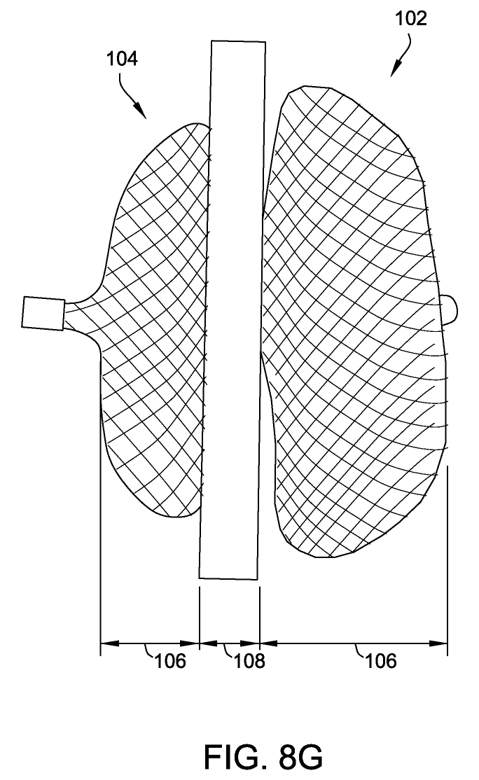

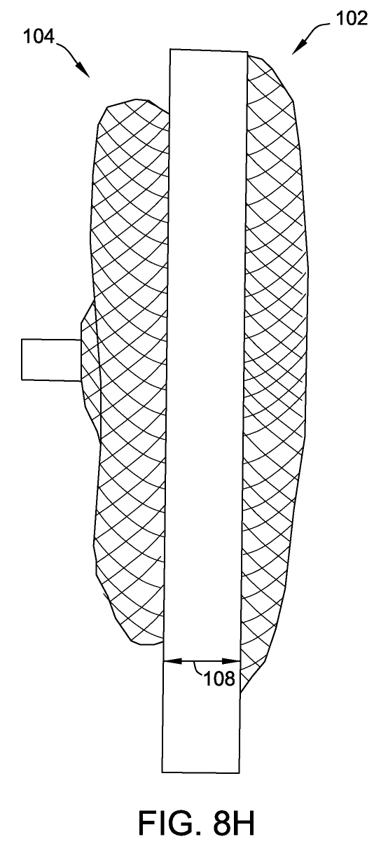

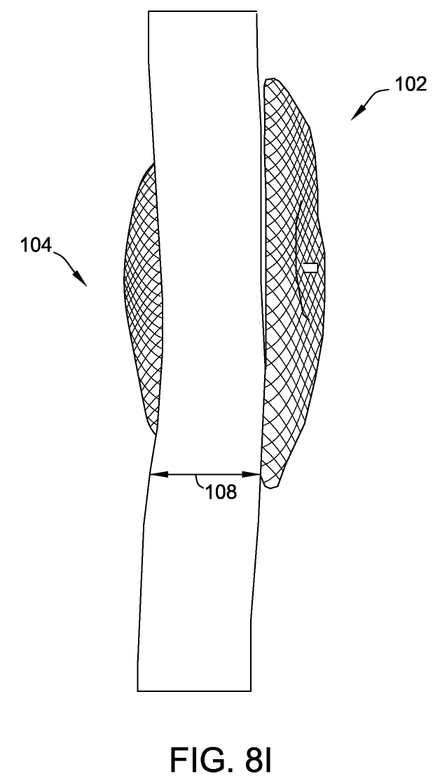

Turning now to FIGS. 8A and 8B, an exemplary embodiment of a locking mechanism system is shown that utilizes a distal locking portion coil 802 comprising a formed wire or low-profile/flat spring that attaches to distal disc portion 102 and pulls from the center of the device through the end screw/threaded release of proximal locking portion 803 thereby securing the discs to one another. Proximal locking portion 803 is threaded to proximal disc portion 104 in order to release from proximal delivery cable 809. In some embodiments, the delivery system can be attached to the distal locking portion coil 802 via proximal delivery cable 809, or a clasping bioptome-like mechanism, or as an attachment element coupled to the end screw of proximal locking portion 803 or proximal disc portion 104. FIG. 8B illustrates the device in a reduced configuration within delivery sheath 812 enabled by elongation of the device. Advantageously, the disc portions 102 and 104 are enabled to pull closer to one another, particularly because very little space is required in the middle of the device to accommodate distal locking portion coil 802 in either the expanded configuration (FIG. 8A) or the reduced configuration (FIG. 8B). Distal locking portion may also be a distal locking portion spring 801 such as a flat coil spring, a flat zig zag pattern spring, a flat clover shaped spring (FIG. 8C), and other suitably shaped springs that unfold during loading into the delivery system and subsequently spring back to shape after deployment. FIGS. 8D, 8E, and 8F show a spring side profile view before heat setting, a spring top view before heat setting, and a spring side view after heat setting and flattening, respectively.

FIG. 8G shows an occluder device as deployed, similar to the known medical device of FIG. 1, without a locking mechanism (i.e., without a spring (e.g., a Nitinol spring) coupling element or other locking mechanism). Bulging 106 of both the distal disc portion 102 and the proximal disc portion 104 is apparent on either side of septum thickness 108.

FIG. 8H shows an occluder device as deployed with a spring coupling element (e.g., a Nitinol spring) as the locking mechanism. In this embodiment, the hole into which the device is deployed is 9 mm; however, the recommended size of the hole is 22 mm based on the size of the device. Consequently, even with an undersized hole/oversized device, bulging 106 has been reduced significantly relative to FIG. 8G by incorporating the spring coupling element locking mechanism.

FIG. 8I shows an occluder device as deployed with a spring coupling element as the locking mechanism. In this embodiment, the hole into which the device is deployed is one-third the recommended size based on the size of the occluder device, and the septal thickness 108 is larger relative to the previous embodiments of FIGS. 8G-8H. A thicker septum is more likely to increase bulging. In the embodiment shown, there is greater device conformity/compliance to the septal wall despite both the increased septal thickness 108 and the undersized hole. Softer occluding devices such as those shown and described in embodiments herein have improved compliance to tissue (such as cardiac tissue) due to the reduced bulging achieved by occluder locking mechanisms.

### c. Criss-cross spring

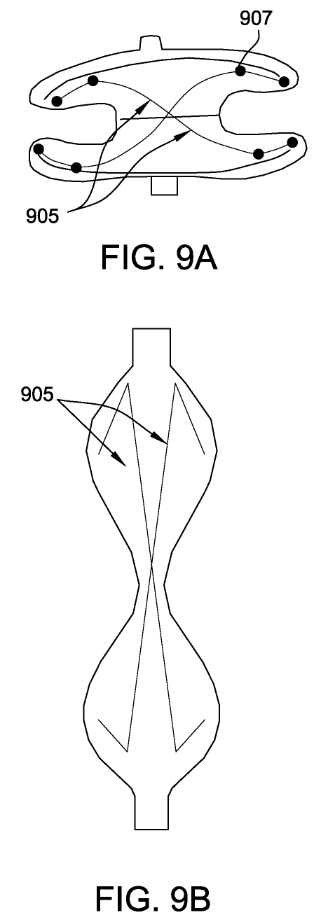

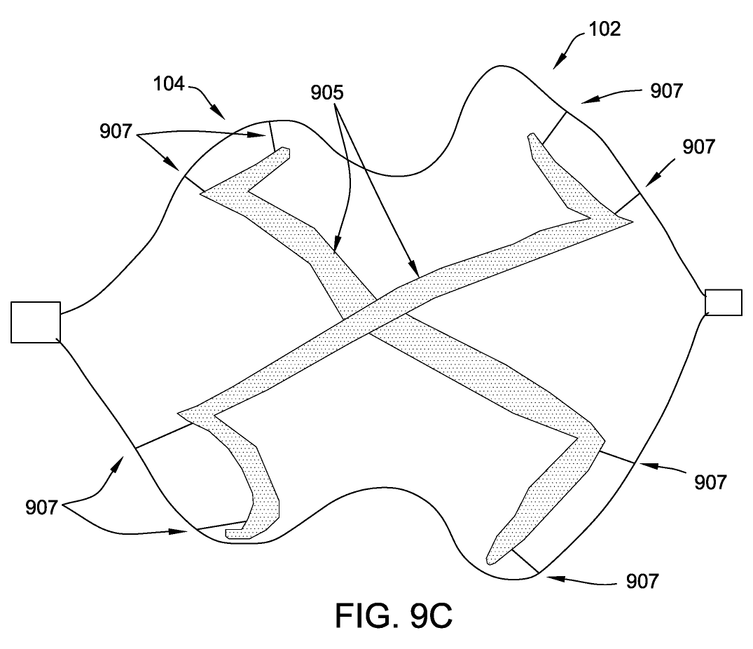

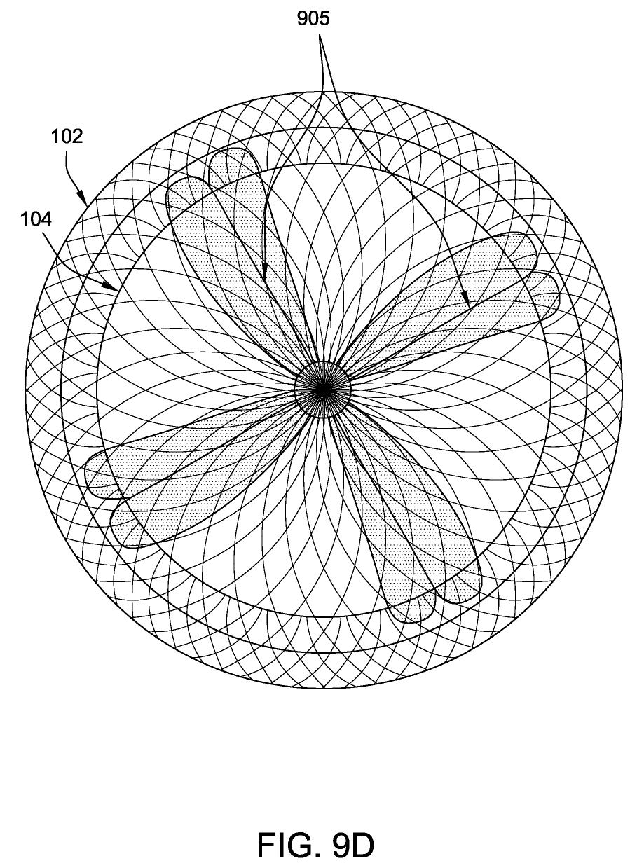

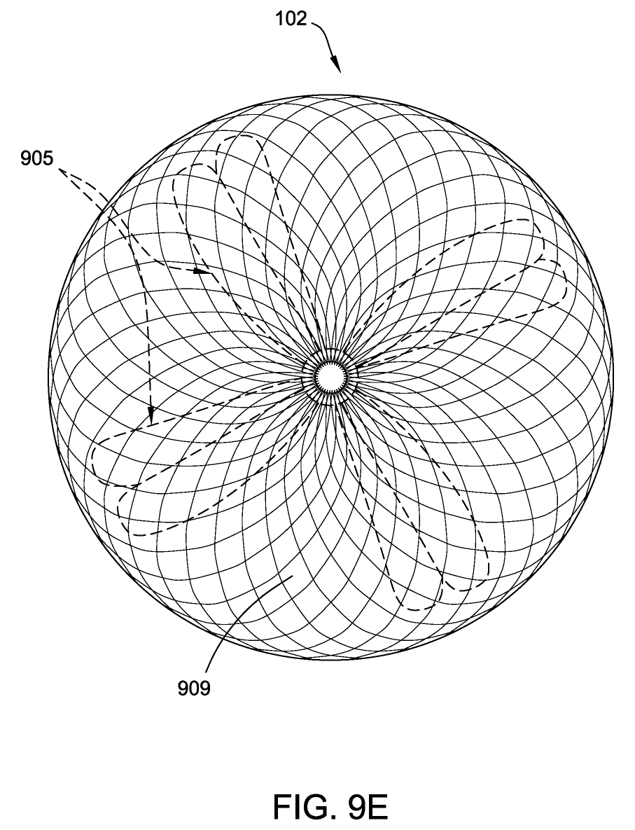

FIGS. 9A-9I show exemplary embodiments of a locking mechanism system with an internal elastomer spring coupling element 905 in accordance with the present disclosure. The elastomer may comprise a material such as Chronoprene or Tecothane^{TM}. Coupling element 905 is internally attached to both distal disc portion and proximal disc portion at attachment points 907 (e.g., sutured attachment points) in a criss-cross type pattern as shown in FIG. 9A such that the pattern allows enough stretch to the elastomer to stretch during delivery (i.e., in the reduced device configuration), yet the elastomer maintains enough strength to pull the disc portions toward each other in the expanded configuration and deployment. When in the reduced configuration (FIG. 9B) the elastomer coupling element 905 stretches to the length of the braid of the device, and when in the expanded configuration (FIG. 9A) the elastomer coupling element 905 reduces bulging of the disc portions due to the criss-cross pattern.

FIG. 9C shows an occluder device in an un-deployed, semi-expanded configuration, including an internal view of elastomer spring coupling element 905 and attachment points 907. FIG. 9D shows a top view of elastomer spring coupling element 905 in an occluder device having no disc portion coverings and in a flattened configuration. These FIGS. (9C and 9D) illustrate how the internal elastomer is attached, as well as the criss-cross configuration. FIG. 9E shows the occluder device of FIG. 9D with PET blood blocking discs 909 inserted into the distal and proximal disc portions.

### d. Methods of using the device

In accordance with the present disclosure, the medical devices disclosed herein are directed toward methods of eliminating or reducing erosion of cardiac tissue. The methods comprise providing a medical device comprising a tubular member comprising a proximal disc portion at a proximal end and a distal disc portion at a distal end and a waist member extending between the proximal disc portion and the distal disc portion; wherein the tubular member has an expanded configuration when deployed at the target site and a reduced configuration for delivery to the target site; and, at least one locking mechanism; constraining the medical device from a preset expanded configuration to a reduced configuration; delivering the medical device; deploying the medical device such that the tubular member returns to the preset expanded configuration; and, increasing the medical device compliance on cardiac tissue.

In some embodiments, the locking mechanism comprises a plurality of locking mechanisms that comprises a plurality of distal locking portions evenly distributed over the distal disc portion and a plurality of proximal locking portions evenly distributed over the proximal disc portion such that each of the plurality of distal locking portions is configured to be coupled to a respective one of the plurality of proximal locking portions. Alternatively, the plurality of locking mechanisms may attach directly to both the distal and proximal disc portions (e.g., no localized distal or proximal locking portions, such as described above for FIGS. 9A-9E) such that they are enabled to pull the distal and proximal disc portions together and effectively maintain the pulled together position of the disc portions.

While embodiments of the present invention have been described, it should be understood that various changes, adaptations and modifications may be made therein without departing from the spirit of the invention and the scope of the appended claims. For example, in view of this disclosure, a person of ordinary skill in the art would recognize the device body portion could be cylindrical, barrel shaped, concave, convex, tapered, or a combination of shapes without departing from the invention herein. Further, all directional references (e.g., upper, lower, upward, downward, left, right, leftward, rightward, top, bottom, above, below, vertical, horizontal, clockwise, and counterclockwise) are only used for identification purposes to aid the reader’s understanding of the present disclosure, and do not create limitations, particularly as to the position, orientation, or use of the disclosure. It is intended that all matter contained in the above description or shown in the accompanying drawings shall be interpreted as illustrative only and not limiting. Changes in detail or structure may be made without departing from the spirit of the disclosure as defined in the appended claims

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A medical device for treating a target site, the medical device comprising:
a tubular member comprising a proximal disc portion at a proximal end, a distal disc portion at a distal end, and a waist member extending between the proximal disc portion and the distal disc portion, wherein the tubular member has an expanded configuration when deployed at the target site and a reduced configuration for delivery to the target site; and
a locking mechanism comprising a distal locking portion attached to the distal disc portion and a proximal locking portion attached to the proximal disc portion, wherein the distal locking portion and the proximal locking portion are configured to be coupled together when the medical device is in the expanded configuration,
wherein the locking mechanism is a passive locking mechanism.

2. The medical device of claim 1, wherein the passive locking mechanism automatically couples the distal locking portion and the proximal locking portion when in the expanded configuration.

3. The medical device of claim 1, wherein the distal locking portion is located in a center of the distal disc portion and wherein the proximal locking portion is located in a center of the proximal disc portion.

4. The medical device of claim 1, wherein the distal locking portion comprises at least one formed loop and wherein the proximal locking portion comprises an end screw.

5. The medical device of claim 4, wherein the at least one formed loop is configured to foreshorten upon deployment of the tubular member into the expanded configuration, the at least one formed loop configured to, upon foreshortening, take a shape within the distal disc portion that pulls the proximal disc portion and the distal disc portion together by forming a second loop on an exterior of the proximal disc portion.

6. The medical device of claim 5, wherein the at least one formed loop is only one formed loop, the only one formed loop configure to form a "U" shape that allows the proximal disc portion and distal disc portion to deploy.

7. The medical device of claim 1, wherein the distal locking portion is a coil comprising a formed wire or a spring that is attached to the distal disc portion.

8. The medical device of claim 1, wherein the distal locking portion is a spring configured to unfold during loading into a delivery system and to subsequently spring back to shape after deployment.

9. The medical device of claim 1, wherein the locking mechanism is reversible such that when the distal locking portion and the proximal locking portion are uncoupled from each other, the distal locking portion remains attached to the distal disc portion and the proximal locking portion remains attached to the proximal disc portion.

10. The medical device of claim 1, wherein the locking mechanism is non-reversible such that when the distal locking portion and the proximal locking portion are uncoupled from each other, at least one of the distal locking portion and the proximal locking portion detaches from its respective disc portion.

11. A medical device for treating a target site, the medical device comprising:
a tubular member comprising a proximal disc portion at a proximal end, a distal disc portion at a distal end, and a waist member extending between the proximal disc portion and the distal disc portion, wherein the tubular member has an expanded configuration when deployed at the target site and a reduced configuration for delivery to the target site; and
a locking mechanism comprising at least one coupling element attached to both the distal disc portion and the proximal disc portion, wherein the at least one coupling element is a spring that internally extends from the distal disc portion to the proximal disc portion in a criss-cross pattern such that the distal disc portion and the proximal disc portion are configured to pull toward each other when the tubular member is in the expanded configuration.

12. The medical device of claim 11, wherein the spring is formed of an elastomer.

13. The medical device of claim 12, wherein in the reduced configuration of the tubular member, the elastomer spring stretches to a length of a braid of the tubular member.

14. The medical device of claim 13, wherein in the expanded configuration of the tubular member, the elastomer spring reduces bulging of the proximal disc portion and the distal disc portion due to the criss-cross pattern.

15. The medical device of claim 11, wherein the cross-cross pattern allows enough stretch to the elastomer spring so that the elastomer spring can stretch when the tubular member is in the reduced configuration.

16. The medical device of claim 15, wherein the elastomer spring maintains enough strength to pull the distal disc portion toward the proximal disc portion when the tubular member is in the expanded configuration.

17. The medical device of claim 11, wherein the spring is attached to the distal disc portion at a distal attachment point, and the spring is attached to the proximal disc portion at a proximal attachment point.

18. The medical device of claim 17, wherein the distal attachment point is a distal suture attachment, and the proximal attachment point is a proximal suture attachment.

19. The medical device of claim 11, wherein the spring is attached to the distal disc portion at a plurality of distal suture attachment points, and the spring is attached to the proximal disc portion at a plurality of proximal suture attachment points.

20. The medical device of claim 11, further comprising polyethylene terephthalate (“PET”) discs in the distal disc portion and in the proximal disc portion.
